(19) United States
(12) Patent Application Publication
(10) Pub. No.: US 2022/0037021 A1
(43) Pub. Date: Feb. 3, 2022

(54) RESOURCE TRACKING AND ALLOCATION

(71) Applicant: T-Mobile USA, Inc., Bellevue, WA (US)

(72) Inventors: ChihChin Wu, Bellevue, WA (US); Boua Lor, Renton, WA (US); Jie Hui, Mercer Island, WA (US); Yasmin Karimli, Kirkland, WA (US)

(21) Appl. No.: 17/390,597

(22) Filed: Jul. 30, 2021

Related U.S. Application Data
(60) Provisional application No. 63/059,626, filed on Jul. 31, 2020.

(51) Int. Cl. G16H 50/20; G16H 40/20; G16H 50/70; G16H 10/60; G16H 40/67

(52) U.S. Cl. CPC G16H 50/20; G16H 40/20; G16H 40/67; G16H 10/60; G16H 50/70

(57) ABSTRACT

Methods, systems, and apparatus, including computer programs encoded on a computer storage medium, for resource tracking and allocation are disclosed. In one aspect, a method includes the actions of receiving data that reflects characteristics of a user. The actions further include receiving data that reflects first medical resources that are available at a first healthcare facility. The actions further include receiving data that reflects second medical resources that are available at a second healthcare facility. The actions further include, based on the user data and medical resource data, determining whether to generate and output a recommendation for the user to visit the first or second healthcare facility. The actions further include, based on the user data and medical resource data, predicting the needs of the first and second healthcare facilities and generating and outputting recommendations to allocate medical resources according to those needs.

### CROSS-REFERENCE TO RELATED APPLICATION

This application claims the benefit of U.S. Application 63/059,626, filed Jul. 31, 2020, which is incorporated by reference.

### BACKGROUND

Healthcare is the maintenance or improvement of health via the prevention, diagnosis, treatment, recovery, or cure of disease, illness, injury, and other physical and mental impairments in people. Healthcare is delivered by health professionals in various health fields. The health professionals may use various pieces of equipment to provide the healthcare.

### BRIEF DESCRIPTION OF THE DRAWINGS

The detailed description is described with reference to the accompanying figures, in which the left-most digit(s) of a reference number identifies the figure in which the reference number first appears. The use of the same reference numbers in different figures indicates similar or identical items.

FIG. 1 illustrates an example system that is configured to generate a recommendation for a user to visit a healthcare facility based on data that reflects the characteristics of the user.

FIG. 2 illustrates an example server that is configured to generate a recommendation for a user to visit a healthcare facility based on data that reflects the characteristics of the user.

FIG. 3 is a flowchart of an example process for generating a recommendation for a user to visit a healthcare facility based on data that reflects the characteristics of the user.

FIG. 4 is a flowchart of an example process for providing a healthcare facility notification of a condition of a person who received a recommendation to visit the healthcare facility.

FIG. 5 is a flowchart of an example process for generating a recommendation for a healthcare facility to transfer medical resources to another healthcare facility.

### DETAILED DESCRIPTION

The techniques as described herein implement resource allocation, specifically healthcare resources. Allocating healthcare resources in an efficient manner may be helpful during times when resources are in short supply because of supply chain disruptions, production disruptions, increased demand, and/or any other similar reason. By allocating healthcare resources in an efficient manner, the healthcare resources may be transported to the locations where they are most needed and/or the locations where they are likely to be needed in the future. Predicting where the healthcare resources are likely to be needed in the future and delivering those resources to those locations will help to mitigate the effects of the shortage.

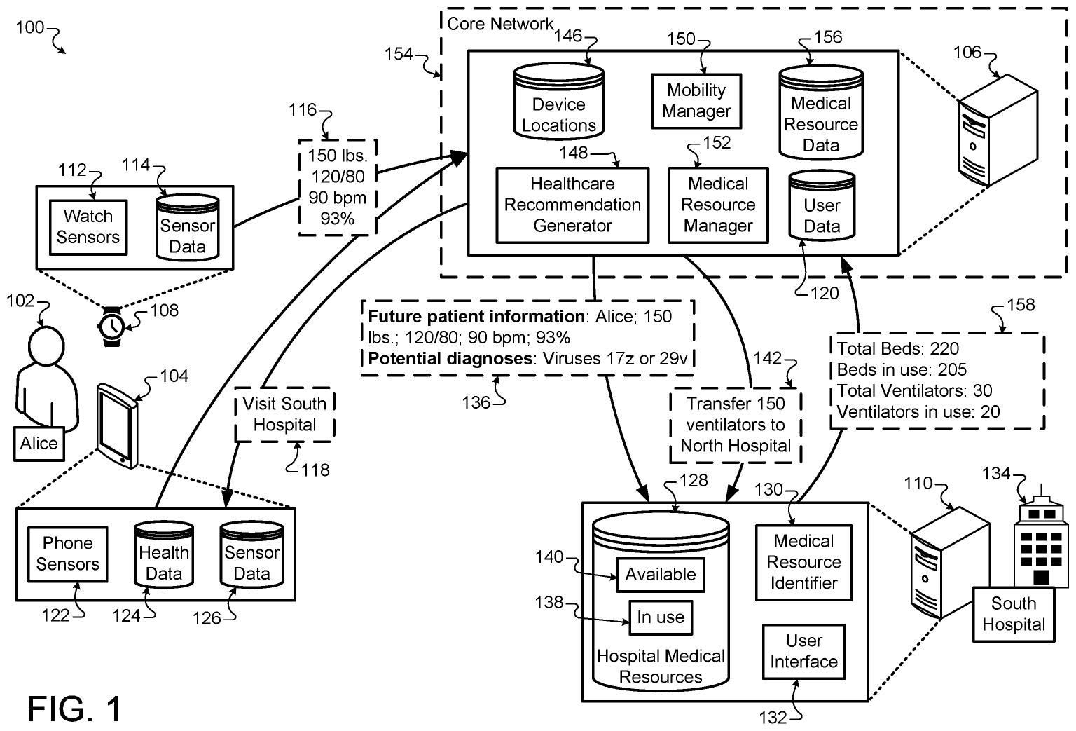

FIG. 1 illustrates an example system 100 that is configured to generate a recommendation for a user 102 to visit a healthcare facility 134 based on data that reflects the characteristics of the user 102. Briefly, and as described in more detail below, the user 102 uses the watch 108 and computing device 104 to collect health-related data. The watch 108 and computing device 104 provide the health-related data to the server 106. The server 106 analyzes the health-related data. Based on the health-related data, the server 106 may recommend that the user 102 visit the hospital 134, quarantine, or take another similar action. In instances where the server 106 recommends that the user 102 visit the hospital 134, the server 106 may provide a hospital server 110 with information related to the user 102.

In more detail, the user 102 may interact with the computing device 104 and the watch 108. The watch 108 may be configured to collect various types of vital signs and other health related data. The watch 108 may include various watch sensors 112 that may automatically collect these vital signs. For example, the watch sensors 112 may be configured with various hardware and/or software components to, among other things, collect blood pressure data, pulse data, blood oxygen data, electrocardiogram data, sleep data, breathing rate data, and/or any other similar health-related data. The watch sensors 112 may store the collected data in the sensor data 114 that may be implemented by a storage device on the watch 108. In some implementations, the watch sensors 112 may include additional sensors such as an ambient light sensor, an accelerometer, an altimeter, proximity sensor, location sensor, gyroscope, ultraviolet light sensor, electrothermal activity sensor, magnetometer, thermometer, and/or any other similar sensor. These additional watch sensors 112 may also store data in the sensor data 114. The watch sensors 112 may store timestamps in the sensor data 114 that indicates when each sensor collected the corresponding data.

The computing device 104 may be any type of computing device that is configured to communicate with other devices through a wired or wireless connection. For example, the computing device 104 may be a mobile phone, tablet, laptop computer, desktop computer, and/or any other similar device. The watch 108 may be any type of quantified self device that is configured to sense and collect data related to a user. The watch 108 may be any type of wearable device such as a fitness tracker, smart glasses, smart watch, step counter, and/or any other similar device.

The user 102 may use computing device 104 to store additional health data 124. The user 102 may enter the health data 124 into the computing device 108. In some implementations, the health data 124 may include data that is similar to the health-related data collected by the watch 108. For example, the user 102 may measure the pulse of the user 102 and enter the pulse into the computing device 104. In some implementations, the health data 124 may include additional health-related data that is different than the health-related data collected by the watch 108. For example, the user 102 may measure the weight of the user 102 and enter the weight into the computing device 104.

The computing device 104 may also include various phone sensors 122. The phone sensors 122 may include a proximity sensor, an accelerometer, an ambient light sensor, a moisture sensor, a gyroscope, a compass, a camera, a microphone, a barometer, location sensor, and/or any other similar sensor. The phone sensors 122 may store the collected data in the sensor data 126 that may be implemented by a storage device on the computing device 104. The phone sensors 122 may store timestamps in the sensor data 126 that indicates when each sensor collected the corresponding data.

In some implementations, the watch 108 and/or computing device 104 may be configured to communicate with external devices. For example, the computing device 104 may communicate with a scale. The scale is configured to transmit weight data to the computing device 104 in response to the scale detecting weight. The computing device 104 may store the weight data in the health data 124. As another example, the watch 108 may communicate with a blood pressure cuff. The blood pressure cuff may be configured to transmit blood pressure data to the watch 108. The watch 108 may store the blood pressure data in the sensor data 114. In some implementations, the sensor data 114, the health data 124, and/or the sensor data 126 may store data indicating the source of the corresponding data. For example, the sensor data 126 may store data indicating that the source of the location data was a GPS sensor on the computing device 104. The sensor data 114 may store data indicating that the source of the blood oxygen data was an external blood oxygen sensor.

The computing device 104 and/or the watch 108 may be configured to provide a user data transmission 116 to the server 106. The user data transmission 116 may include data stored in the sensor data 114, the health data 124, and/or the sensor data 126. The computing device 104 and/or the watch 108 may provide the user data transmission 116 at periodic intervals, such as every hour. The computing device 104 and/or the watch 108 may provide the user data transmission 116 to the server 106 in response to a request from the server 106 or another computing device. The computing device 106 and/or the watch 108 may provide the user data transmission 116 to the server 106 in response to additional data being stored in the sensor data 114, the health data 124, and/or the sensor data 126.

In some implementations, the computing device 104 may provide the user data transmission 116 to the server 106. In this case, the watch 108 may provide the sensor data 114 to the computing device 104. The user data transmission 116 may include the health data 124, the sensor data 126, and the sensor data 114 received from the watch 108. In some implementations, the watch 108 may provide the user data transmission to the server 106. In this case, the computing device 104 may provide the sensor data 126 and the health data 124 to the watch 108. The user data transmission 116 may include the health data 124, the sensor data 126, and the sensor data 114 received from the computing device 104. In some implementations, the computing device 104 and the watch 108 may each provide a separate user data transmission 116 to the server 106.

The server 106 may receive the user data transmission 116 and store the user data transmission 116 in the user data 120 that is implemented in a storage device of or accessible by the server 106. The user data 120 may include the user data transmission 116, data identifying a device that provided the user data transmission 116, and/or a timestamp indicating when the server 106 received the user data transmission 116. The server 106 may be included in or in communication with a network such as a wireless carrier network that provides voice and data communication services to multiple devices, such as the computing device 104, the watch 108, and/or the hospital server 110 and other devices. The wireless carrier network may provide telecommunication and data communication in accordance with one or more technical standards, such as Enhanced Data Rates for GSM Evolution (EDGE), Wideband Code Division Multiple Access (W-CDMA), High Speed Packet Access (HSPA), Long Term Evolution (LTE), 5th Generation (5G) wireless systems, CDMA-2000 (Code Division Multiple Access 2000), and/or other similar standards. In some implementations, the server 106 may communicate with the computing device 104, the watch 108, and/or the hospital server 110 and other devices using a Wi-Fi network, short range radio, infrared communication, and/or any other similar communication technique.

The wireless carrier network may include a radio access network and a core network 154. The radio access network may include multiple base stations. The multiple base stations are responsible for handling voice and/or data traffic between multiple devices, such as the computing device 104, the watch 108, and/or the hospital server 110 and other devices and the core network 154. Accordingly, each of the base stations may provide a corresponding network cell that delivers telecommunication and data communication coverage. The core network 154 may use the network cells to provide communication services to the multiple subscriber devices. For example, the core network 154 may connect the multiple devices to other telecommunication and data communication networks, such as the Internet and the public switched telephone network (PSTN). The base stations are responsible handling voice and data traffic between devices and the core network 154. In some implementations, the base stations may be in the form of eNodeB nodes. Each eNodeB node may include a base transceiver system (BTS) that communicates via an antenna system over an air-link with one or more devices that are within range. The antenna system of an eNodeB node may include multiple antennas that are mounted on a radio tower to provide a coverage area that is referred to as a “cell.” The BTS may send radio signals to devices and receive radio signals from devices.

The server 106 may include a mobility manager 150. The mobility manager 150 may be configured to monitor the location of the computing device 104 and the watch 108 that are connected to the server 106 through a wireless base station. The location of the computing device 104 and the watch 108 may include the location of the wireless base station to which the computing device 104 and the watch 108 are connected and/or GPS data received from the computing device 104 and the watch 108. The mobility manager 150 may store the location data in the device locations 146 of the server 106.

In some implementations, the mobility manager 150 may determine the location of a computing device 104 and the watch 108 at periodic intervals, such as every five seconds. In some implementations, the mobility manager 150 may determine the location of a computing device 104 and the watch 108 when the computing devices 106 and 108 connect to a different wireless base station and/or provide updated GPS data. In some implementations, the mobility manager 150 may determine the location of the computing device 104 and the watch 108 relative to the base station with which the computing device is communicating. In this case, the mobility manager 150 may determine the relative location based on data collected from the base station such as signal strength and direction of communications between the computing device 104 and the watch 108 and the base station. The mobility manager 150 may also determine the relative location based on the location of the base station and GPS data received from the computing device 104 and the watch 108. The relative location data may include a distance between the computing device 104 and the watch 108 and the base station, the cardinal direction from the base station to the computing device 104 and the watch 108, and/or any other similar measurements.

The server 106 may be configured to communicate with the hospital server 110 of the hospital 134. The hospital server 110 may be configured to identify and store data related to the medical resources located and in use at the hospital 134. The medical resources may include reusable medical resources such as beds, ventilators, vital sign monitoring devices, and/or other similar types of reusable resources. The medical resources may also include consumable medical resources such as medications, vaccines, bandages, and/or other similar types of consumable resources.

The hospital server 110 may receive data related to the medical resources in various manners. The hospital server 110 may include a user interface 132. The user interface 132 may be configured to receive information from a user. The information may include data related to the quantity of available medical resources and medical resources in use. For example, a user may provide data indicating that one hundred doses of a vaccine are available. The user may also provide data indicating that twenty vaccines were administered. As another example, a user may provide data indicating that ten beds are available, and one hundred fifty beds are in use. The hospital server 110 may store the received data in the hospital medical resources 128. The hospital medical resources 128 may be implemented by a storage device of the hospital server 110. The hospital medical resources 128 may include availability data 140 that indicates the medical resources that are available and in-use data 138 that indicates the medical resources that are being used by a patient. In some implementations, the availability data 140 may include data related to reusable and consumable medical resources and the in-use data 138 may include data related to reusable medical resources.

The hospital server 110 may also include a medical resource identifier 130. The medical resource identifier 130 may be configured with various hardware and/or software components to, among other things, communicate with various medical resources that may be equipped with a communications interface. For example, a bed may include a pressure sensor and a communications interface. The communications interface of the bed may provide data to the medical resource identifier 130 indicating whether the pressure sensor detects pressure in the bed. Based on that information, the medical resource identifier 130 may store data in the hospital medical resources 128 indicating whether the bed is available or in use. As another example, a ventilator may include a communications interface. The communications interface of the ventilator may provide data to the medical resource identifier 130 indicating whether ventilator is in use. Based on that information, the medical resource identifier 130 may store data in the hospital medical resources 128 indicating whether the ventilator is available or in use.

Some medical resources may not include communications interfaces. In this case, some of those medical resource may be stored on, stored in, or located nearby devices that are configured to communicate with the hospital server 110. For example, bandages may be stored on various scales throughout the hospital 134. The scales may be configured with various hardware and/or software components to, among other things, communicate with the medical resource identifier 130 and provide data indicating the weight detected by the scale. The medical resource identifier 130 may compare the weight detected by the scale and the weight of the bandages stored on that scale. Based on that comparison, the medical resource identifier 130 may determine the number of bandages that are available and store that data in the hospital medical resources 128.

The hospital server 110 may be configured to provide medical resource data transmission 158 to the server 110. The medical resource data transmission 158 may include data stored in the hospital medical resources 128. The hospital server 110 may provide the medical resource data transmission 158 at periodic intervals, such as every hour. The hospital server 110 may provide the medical resource data transmission 158 in response to a request from the server 106 or another computing device. The hospital server 110 may provide the medical resource data transmission 158 to the server 106 in response to a change in the hospital medical resources 128.

The server 106 may receive the medical resource data transmission 158 and store the medical resource data transmission 158 in the medical resource data 156 that is implemented in a storage device of the server 106. The medical resource data 156 may include the medical resource data transmission 158, data identifying a device or hospital that provided the medical resource data transmission 158, and/or a timestamp indicating when the server 106 received the medical resource data transmission 158.

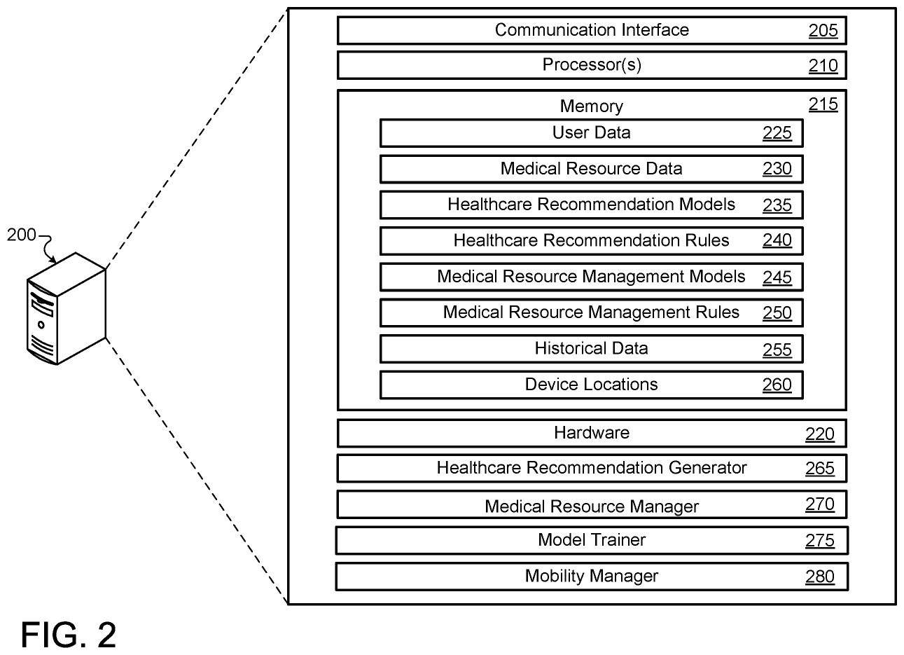

The server 106 may include a healthcare recommendation generator 148. The healthcare recommendation generator 148 may be configured to analyze the user data 120, the medical resource data 156, and/or the device locations 146 and generate a recommended action for the user 102. The recommended action may be a health-related action such as a recommendation to visit a hospital, a recommendation to quarantine, a recommendation to call an emergency number, and/or any other similar health-related action. The healthcare recommendation generator 148 may use various models and/or rules to analyze the user data 120, the medical resource data 156, and/or the device locations 146. The rules and models will be discussed in more detail below in relation to FIG. 2.

The healthcare recommendation generator 148 may generate a user recommendation 118 based on the user data 120, the medical resource data 156, and/or the device locations 146. In some implementations, the healthcare recommendation generator 148 may analyze the user data 120 that corresponds to the user 102, the device locations 146 that correspond to the computing device 104 and watch 108, and the medical resource data 156. In some implementations, the healthcare recommendation generator 148 may analyze the user data 120 that corresponds to the user 102 and other users, the device locations 146 that correspond to the computing device 104 and watch 108 and the devices of other users, and the medical resource data 156.

As an example, the healthcare recommendation generator 148 may analyze the user data 120, the medical resource data 156, and/or the device locations 146 and generate the user recommendation 118 that recommends that the user 102 visit the hospital 134. The server 106 may provide the user recommendation 118 to the computing device 104. The computing device 104 may output the user recommendation 118. The user 102 may determine to comply with the recommendation or ignore the user recommendation 118. In some implementations, the user 102 may respond to the user recommendation 118 with an indication whether the user 102 will comply or ignore the user recommendation 118. In some implementations, the healthcare recommendation generator 148 may access the device locations 146 to determine the locations of the watch 108 and/or the computing device 104 to infer whether the user 102 complied with or ignored the user recommendation 118.

In conjunction with generating the user recommendation 118, the healthcare recommendation generator 148 may generate a hospital notification 136. The hospital notification 136 may include medical data related to the user 102 such as the data received from the computing device 104 and the watch 108 and an identity of the user 102. The hospital notification 136 may also include a potential diagnosis as determined by the health recommendation generator 148. Based on the user data 120, the medical resource data 156, and/or the device locations 146, the healthcare recommendation generator 148 may determine the likely diagnosis using the models and/or rules used to determine the user recommendation 118. In some implementations, the healthcare recommendation generator 148 may generate the hospital notification 136 if the user recommendation 118 indicates to go to the hospital. In this case, the healthcare recommendation generator 148 may provide the hospital notification 136 to the server of the hospital indicated in the user recommendation 118.

The server 106 may include a medical resource manager 152. The medical resource manager 152 may be configured with various hardware and/or software to, among other things, analyze the user data 120, the medical resource data 156, and/or the device locations 146 to determine resource recommendations 142 to provide to hospitals. The resource recommendations 142 may include instructions for redistributing medical resources located at various hospitals. The medical resource manager 152 may generate the resource recommendations 142 using various rules and/or models that will be discussed in more detail below in relation to FIG. 2.

In some implementations, the medical resource manager 152 may be configured to analyze the user recommendations and hospital notifications generated by the healthcare recommendation generator 148 in addition to the user data 120, the medical resource data 156, and/or the device locations 146. In some implementations, the generation of the user recommendation 118 and/or the hospital notification 136 may trigger the medical resource manager 152 to analyze the user recommendation 118, the hospital notification 136, the user data 120, the medical resource data 156, and/or the device locations 146. In this case, the medical resource manager 152 may ensure that the hospital identified in the hospital notification 136 is likely to have the necessary equipment to treat the user 102.

In some implementations, the medical resource manager 152 may analyze the user recommendation 118, the hospital notification 136, the user data 120, the medical resource data 156, and/or the device locations 146 if the user recommendation 118 includes a recommendation for the user 102 to visit a hospital. If the user recommendation 118 includes a recommendation not to visit a hospital, then the medical resource manager 152 may not analyze the user recommendation 118, the hospital notification 136, the user data 120, the medical resource data 156, and/or the device locations 146.

In some implementations, the medical resource manager 152 may analyze the medical resource data transmission 158 and determine that some medical resources are all in use, a threshold amount are in use, or there is less than a threshold amount remaining. For example, the medical resource data transmission 158 may indicate that the ventilators at the hospital 134 are all in use. In response to this determination, the medical resource manager 152 may analyze the user data 120, the medical resource data 156, and/or the device locations 146 and generate a resource recommendation 142. As another example, the medical resource data transmission 158 may indicate that the number of doses of a medication is below a threshold. In response to this determination, the medical resource manager 152 may analyze the user data 120, the medical resource data 156, and/or the device locations 146 and generate a resource recommendation 142.

The server 110 may receive the resource recommendation 142, and the user interface 132 may output the resource recommendation 142 on a display of the server 110 and/or a computing device connected to the server 110. A user may view the resource recommendation 142 and indicate whether the user intends to comply with or reject the resource communication 142. The medical resource manager 152 may receive the comply or reject indication and use that indication as an input for the next analysis of the user data 120, the medical resource data 156, and/or the device locations 146. In some implementations, the medical resource manager 152 may analyze an additional medical resource data transmission 158 to determine whether the hospital complied with the resource recommendation 142.

The healthcare recommendation generator 148 and the medical resource manager 152 may continue to analyze the user data 120, the medical resource data 156, and/or the device locations 146 as the server 106 receives additional user data transmissions 116 and additional medical resource data transmissions 158. The healthcare recommendation generator 148 may continue to generate additional user recommendations 118, additional hospital notifications 136, and/or additional resource recommendations 142.

FIG. 2 illustrates an example server 200 that is configured to generate a recommendation for a user to visit a healthcare facility based on data that reflects the characteristics of the user. The server 200 may be any type of computing device that is configured to communicate with other computing devices. The server 200 may be integrated into a wireless carrier network or interact with a wireless carrier network. The server 200 may communicate with other computing devices using a wide area network, a local area network, the internet, a wired connection, a wireless connection, and/or any other type of network or connection. The wireless connections may include Wi-Fi, short-range radio, infrared, and/or any other wireless connection. The server 200 may be similar to the server 106 of FIG. 1. Some of the components of the server 200 may be implemented in a single computing device or distributed over multiple computing devices. For example, components of the server 200 may be implemented in the server 106, the computing device 104, the watch 108, and/or the hospital server 110. Some of the components may be in the form of virtual machines or software containers that are hosted in a cloud in communication with disaggregated storage devices.

The server 200 may include a communication interface 205, one or more processors 210, memory 215, and hardware 220. The communication interface 205 may include communication components that enable the server 200 to transmit data and receive data from devices connected to the wireless carrier network. The communication interface 205 may include an interface that is configured to communicate with base stations of a wireless carrier network. The communication interface 205 may receive data that other devices transmit to the base stations and/or transmit data to the base stations for transmission to the other devices. In some implementations, the communication interface 205 may be configured to communicate over a wide area network, a local area network, the internet, a wired connection, a wireless connection, and/or any other type of network or connection. The wireless connections may include Wi-Fi, short-range radio, infrared, and/or any other wireless connection.

The hardware 220 may include additional user interface, data communication, or data storage hardware. For example, the user interfaces may include a data output device (e.g., visual display, audio speakers), and one or more data input devices. The data input devices may include, but are not limited to, combinations of one or more of keypads, keyboards, mouse devices, touch screens that accept gestures, microphones, voice or speech recognition devices, and any other suitable devices.

The memory 215 may be implemented using computer-readable media, such as computer storage media. Computer-readable media includes, at least, two types of computer-readable media, namely computer storage media and communications media. Computer storage media includes volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device. In contrast, communication media may embody computer-readable instructions, data structures, program modules, or other data in a modulated data signal, such as a carrier wave, or other transmission mechanism. In some implementations, the data stored in the memory 215 may stored externally from the server 200.

The one or more processors 210 may implement a mobility manager 280 in software stored in the memory 215. The mobility manager 280 may be similar to the mobility manager 150 of FIG. 1. The mobility manager 280 may be configured to monitor the location of a computing device that is connected to the server 200 through a wireless base station. The location of the computing device may include the location of the wireless base station to which the computing device is connected and/or GPS data received from the computing device. The mobility manager 280 may store the location data in the device locations 260 of the server 200 in the memory 215.

In some implementations, the mobility manager 280 may determine the location of a computing device at periodic intervals, such as every five seconds. In some implementations, the mobility manager 280 may determine the location of a computing device when the computing device connects to a different wireless base station and/or provides updated GPS data. In some implementations, the mobility manager 280 may determine the location of the computing device relative to the base station with which the computing device is communicating. In this case, the mobility manager 280 may determine the relative location based on data collected from the base station such as signal strength and direction of communications between the computing device and the base station. The mobility manager 280 may also determine the relative location based on the location of the base station and GPS data received from the computing device. The relative location data may include a distance between the computing device and the base station, the cardinal direction from the base station to the subscriber device, and/or any other similar measurements.

The memory 215 may store user data 225. The user data 225 may be similar to the user data 120 of FIG. 1. The user data 225 may store data received from various computing devices of the users. The user data 225 may include sensor data received from the various sensors of the computing devices, data received from other devices that communicate with the computing devices, and/or data received from the users of the computing devices. The user data 225 may include health-related data that may be collected from various health related sensors of the computing devices.

The memory 215 may store medical resource data 230. The medical resource data 230 may be similar to the medical resource data 156 of FIG. 1. The medical resource data 230 may store data received from various hospitals. The medical resource data 230 may include data related to the amount and type of reusable medical equipment located at each hospital and whether that medical equipment is in use. The medical resource data 230 may also include data related to the amount and type of consumable medical equipment, medicines, and/or vaccines located at each hospital.

The one or more processors 210 may implement the healthcare recommendation generator 265 in software stored in the memory 215. The healthcare recommendation generator 265 may be similar to the healthcare recommendation generator 148 of FIG. 1. The healthcare recommendation generator 265 may be configured to analyze the user data 225, the medical resource data 230, and/or the device locations 260 and generate a recommended action for the user. The recommended action may be a health-related action such as a recommendation to visit a hospital, a recommendation to quarantine, a recommendation to call an emergency number, and/or any other similar health-related action. The healthcare recommendation generator 265 may use the healthcare recommendation models 235 and/or the healthcare recommendation rules 240 to analyze the user data 225, the medical resource data 230, and/or the device locations 260.

The one or more processors 210 may implement the model trainer 275 in software stored in the memory 215. The model trainer 275 may be configured to generate the healthcare recommendation models 235 and/or the healthcare recommendation rules 240 by analyzing the historical data 255 in the memory 215. The historical data 255 may include past data similar to the user data 225, the medical resource data 230, and/or the device locations 260 along with data related to medical related actions taken by users and the health condition of those users. Examples of some of the types of past data that may be included in the historical data 255 include weight data, blood pressure data, pulse data, blood oxygen data, electrocardiogram data, accelerometer data, location data, audio data, image data, proximity sensor data, ambient light data, gravity sensor data, gyroscope data, breathing rate, body temperature, ambient temperature, and/or any other similar data. The historical data 255 may also include data related to a medical diagnosis of a user, whether the user is quarantining, whether the user is located at a hospital, if the user is in the hospital then the part of the hospital that the user is located, reusable medical resources being used by the user, consumable medical resources used by the user including medications and vaccines, and/or any other similar data. The historical data 255 may also include the reusable medical resources located at one or more hospitals, the locations of the one or more hospitals, the reusable medical resources being used at one or more hospitals, the consumable medical resources being used at one or more hospitals, data indicating a number and type of reusable or consumable medical resources requested but not available at one or more hospitals, and/or any other similar data. The historical data 255 may also include timestamp data that indicates a date and/or time when the corresponding data was collected and user identification data and/or hospital identification data that indicates to which user or hospital any data corresponds.

The model trainer 275 may analyze the historical data 255 to identify patterns for generating the healthcare recommendation rules 240. The model trainer 275 may identify user data that corresponds to different medical diagnoses and/or whether the user is quarantining or has been admitted to a hospital. For example, the model trainer 275 may identify a pattern that indicates that users who have a blood oxygen level below ninety four percent and a pulse above ninety beats per minute have been diagnosed with virus 17z seventy percent of the time. The model trainer 275 may identify another pattern that indicates that users who have a blood oxygen level between ninety-four and ninety-six and a pulse between eighty and ninety beats per minute have been diagnosed with virus 17z forty percent of the time. The model trainer 275 may generate a rule that indicates that if a user's pulse is above eighty and blood oxygen level if below ninety four percent, then the user should receive a recommendation to travel to a hospital. The model trainer 275 may generate another rule that indicates that if a user's blood oxygen level is between ninety-four and ninety-six and pulse is between eighty and ninety beats per minute, then the user should receive a recommendation to quarantine.

In some implementations, the healthcare recommendation rules 240 may include user-specified rules. These rules may be ones that indicate a pattern to identify in the user data and specify an action to recommend to the corresponding user. For example, a user-specified rule may indicate that if a user has a seventy percent likelihood of having virus 29v and the closest hospital has a bed and ventilator available, then the user should receive a recommendation to visit that hospital. Another user-specified rule may indicate that if a user has a seventy percent likelihood of having virus 29v and the closest hospital does not have a bed and equipment available, then the user should receive a recommendation to quarantine. Another user-specified rule may indicate that if a user has an eighty percent likelihood of having virus 29v and the closest hospital does not have a bed and equipment available and the next closest hospital does have a bed and equipment available, then the user should receive a recommendation to visit the second closest hospital.

The healthcare recommendation rules 240 may include additional user-specified rules. For example, a user-specified rule may indicate that if a user likely has a condition that requires certain medical resources to treat, then the user should receive a recommendation to visit the hospital that has those specific medical resources available. If there are more than one hospital that have those specific medical resource available, then the user should receive a recommendation to visit the hospital that has the shortest combined wait time and travel time. For example, three of ten hospitals within a threshold distance of the user have the specific medical resources. The first hospital is ten minutes away and has a wait time of thirty minutes. The second hospital is twenty minutes away and has a wait time of ten minutes. The third hospital is forty minutes away and has a wait time of fifteen minutes. The second hospital has the lowest combined wait and travel time of thirty minutes, so the user should receive a recommendation to visit the second hospital.

The model trainer 275 may analyze the historical data 255 to generate the healthcare recommendation models 235. The model trainer may train the healthcare recommendation models 235 using machine learning and the historical data. The model trainer 275 may generate various data samples that the model trainer 275 may use to train the healthcare recommendation models 235. Each group of data samples may include similar types of data, and the model trainer 275 may use each group to train a healthcare recommendation model that is configured to receive and/or output different types of data.

The model trainer 275 may analyze the historical data 255 to generate the data samples. Each data sample may represent a point in time for a user. The model trainer 275 may generate multiple data samples for the same user for different points in time. As an example, the historical data 255 may include blood oxygen data, electrocardiogram data, accelerometer data, location data, data related to a medical diagnosis, data indicating whether the user is located at a hospital, data indicating whether the user is quarantining, and reusable and consumable medical resources located and available at the hospital and/or one or more nearby hospitals. The model trainer 275 may generate multiple data samples that represent the values of these data fields at different times, such as every six hours for the period of time when this data is available for that user. The model trainer 275 may generate additional data samples that include the same type of data using data from different users. The additional data samples may correspond to the same or different times. Using these data samples and machine learning, the model trainer 275 may train a healthcare recommendation model. The resulting model may be configured to receive blood oxygen data, electrocardiogram data, accelerometer data, location data, and reusable and consumable medical resources located and available at one or more nearby hospitals. The model may be configured to output data indicating a likely diagnosis of the user and data indicating whether the user should quarantine, visit one of the nearby hospitals, or take no action.

The model trainer 275 may generate additional data samples using the historical data 255. The model trainer 275 may use these models to train additional models that are configured to receive various combinations of inputs for a user. These inputs may include weight data, blood pressure data, pulse data, blood oxygen data, electrocardiogram data, accelerometer data, location data, audio data, image data, proximity sensor data, ambient light data, gravity sensor data, gyroscope data, breathing rate, body temperature, ambient temperature, reusable medical resources being used by the user, consumable medical resources used by the user including medications and vaccines, reusable medical resources located at and being used at one or more hospitals, the consumable medical resources being used at and available at the one or more hospitals, and data indicating a number and type of reusable or consumable medical resources requested but not available at the one or more hospitals. The models may be configured to output various types of outputs. These outputs may include a likely medical diagnosis of the user, a recommendation for the user to quarantine, and/or a recommendation for the user to visit one of the nearby hospitals.

The healthcare recommendation generator 265 may be configured to access the user data 225, the medical resource data 230, and/or the device locations 260 and determine the type of data included in the user data 225, the medical resource data 230, and/or the device locations 260 for a particular user. The healthcare recommendation generator 265 may determine whether any rules in the healthcare recommendation rules 240 specify how to analyze the types of data in the user data 225, the medical resource data 230, and/or the device locations 260 of the particular user. If the healthcare recommendation generator 265 identifies a rule that specifies how to analyze the user data 225, the medical resource data 230, and/or the device locations 260, then the healthcare recommendation generator 265 may apply that rule and generate and output the recommendation specified by the rule.

The healthcare recommendation generator 265 may also determine whether any models in the healthcare recommendation models 235 are configured to receive the user data 225, the medical resource data 230, and/or the device locations 260 for the particular user. If the healthcare recommendation generator 265 identifies a model that is configured to receive the user data 225, the medical resource data 230, and/or the device locations 260 for the particular user, then the healthcare recommendation generator 265 may provide the data as an input to the model. The model may output a likely medical diagnosis of the user, a recommendation for the user to quarantine, and/or a recommendation for the user to visit a nearby a hospital. The healthcare recommendation generator 265 may generate and output a recommendation based on the output of the model.

The one or more processors 210 may implement the medical resource manager 270 in software stored in the memory 215. The medical resource manager 270 may be similar to the medical resource manager 152 of FIG. 1. The medical resource manager 270 may be configured to analyze the user data 225, the medical resource data 230, and/or the device locations 260 and generate a recommended action for a hospital to provide medical resources to another hospital. The recommended action may be a recommendation to provide reusable medical resources, provide consumable medical resources, and/or any other similar action. The medical resource manager 270 may use the medical resource management models 245 and/or the medical resource management rules 250 to analyze the user data 225, the medical resource data 230, and/or the device locations 260.

The model trainer 275 may analyze the historical data 255 to identify patterns for generating the medical resource management rules 250. The model trainer 275 may identify medical resources that may typically go underutilized at a first hospital while similar medical resources at a second hospital may run out. Based on this pattern, the model trainer 275 may generate rules for recommending the transfer of certain medical resources from the first hospital to the second hospital. For example, the model trainer 275 may analyze the historical data 255 and determine that a first hospital typically uses at least ninety percent of its ventilators and a second hospital typically uses around fifty percent of its ventilators. In this case, the model trainer 275 may generate a rule that recommends the second hospital transfer twenty percent of its ventilators if the usage rate is less than fifty percent. As another example, the model trainer 275 may analyze the historical data 255 and determine that a first hospital typically runs out of saline intravenous bags when there are at least fifty users within a three mile radius of the first hospital who have a pulse below sixty beats per minute, breathing rate below fifteen breaths per minute, and blood pressure below 110/70. The model trainer 275 may generate a rule that a second hospital outside of a ten mile of the first hospital provide twenty saline intravenous bags to the first hospital when there are at least fifty users within a three mile radius of the first hospital who have a pulse below sixty beats per minute, breathing rate below fifteen breaths per minute, and blood pressure below 110/70.

In some implementations, the medical resource management rules 250 may include user-specified rules. These rules may be ones that indicate a pattern to identify in the user data 225, medical resource data 230, and/or device locations 260 and specify an action for a hospital to share medical resources. For example, a user-specified rule may indicate to transfer twenty doses of a vaccine for Virus 37w from a first hospital to a second hospital if the first hospital has less than ten doses and the second hospital has more than one hundred doses. Another user-specified rule may indicate to transfer ventilators among three different hospitals to ensure that each hospital has an equal number of unused ventilators. The rule may identify the number of unused ventilators and generate recommendations to transfer ventilators so that each hospital has an equal number of unused ventilators. Another user-specified rule may indicate to transfer ventilators among ten different hospitals so that a first hospital has at least twenty unused ventilators if there are at least fifty people within a three mile radius of the hospital that have receive quarantining recommendations.

The model trainer 275 may analyze the historical data 255 to generate the medical resource management models 245. The model trainer 275 may train the medical resource management models 245 using machine learning and the historical data 255. The model trainer 275 may generate various data samples that the model trainer 275 may use to train the medical resource management models 245. Each group of data samples may include similar types of data, and the model trainer 275 may use each group to train a medical resource management model that is configured to receive and/or output different types of data.

The model trainer 275 may analyze the historical data 255 to generate the data samples. Each data sample may represent a point in time for a hospital. The model trainer 275 may generate multiple data samples for the same hospital for different points in time. As an example, the historical data 255 may include the reusable medical resources located at and being used at one or more hospitals, the consumable medical resources being used at and available at one or more hospitals, and a number and type of requested medical resources that one or more hospitals do not have. The historical data 255 may also include user data related to the users at or nearby the one or more hospitals. The user data may include blood oxygen data, electrocardiogram data, accelerometer data, location data, data related to a medical diagnosis, data indicating whether the user is located at a hospital, and data indicating whether the user is quarantining. The model trainer 275 may generate multiple data samples that represent the values of the medical resource data fields for and the user fields at different times, such as every six hours for the period of time when the data is available. The model trainer 275 may generate additional data samples that include the same type of data using data using different groups of hospitals. The additional data samples may correspond to the same or different times.

Using these data samples and machine learning, the model trainer 275 may train a medical resource management model. The resulting model may be configured to receive the reusable medical resources located at and being used at one or more hospitals, the consumable medical resources being used at and available at the one or more hospitals, blood oxygen data of one or more users near the one or more hospitals, electrocardiogram data of the one or more users, accelerometer data of the one or more users, location data of the one or more users, data related to a medical diagnosis of the one or more users, data indicating whether the one or more users are located at the one or more hospitals, and data indicating whether the one or more users are quarantining. The model may be configured to output data indicating recommendations for one or more of the medical resources to transfer between the one or more hospitals.

The model trainer 275 may generate additional data samples using the historical data 255. The models may be configured to receive various combinations of inputs related to one or more hospitals. These inputs may include reusable medical resources located at and being used at one or more hospitals, the consumable medical resources being used at and available at the one or more hospitals, user data for those users within a threshold distance of the one or more hospitals including weight data, blood pressure data, pulse data, blood oxygen data, electrocardiogram data, accelerometer data, location data, audio data, image data, proximity sensor data, ambient light data, gravity sensor data, gyroscope data, breathing rate, body temperature, and ambient temperature. The models may be configured to output various types of outputs. These outputs may include a recommendation for a hospital to request medical resources from a supplier, and/or a recommendation for two or more hospitals to exchange medical resources.

The medical resource manager 270 may be configured to access the user data 225, the medical resource data 230, and/or the device locations 260 and determine the type of data included in the user data 225, the medical resource data 230, and/or the device locations 260 for one or more hospitals. The medical resource manager 270 may determine whether any rules in the medical resource management rules 250 specify how to analyze the types of data in the user data 225, the medical resource data 230, and/or the device locations 260 of one or more hospitals. If the medical resource manager 270 identifies a rule that specifies how to analyze the user data 225, the medical resource data 230, and/or the device locations 260, then the medical resource manager 270 may apply that rule and generate and output the recommendation specified by the rule.

The medical resource manager 270 may also determine whether any models in the medical resource management models 245 are configured to receive the user data 225, the medical resource data 230, and/or the device locations 260 related to one or more hospitals. If the medical resource manager 270 identifies a model that is configured to receive the user data 225, the medical resource data 230, and/or the device locations 260 for the one or more hospitals, then the medical resource manager 270 may provide the data as an input to the model. The model may output a recommendation for the one or more hospitals to request medical resources from a supplier and/or a recommendation for at least two of the one or more hospitals to exchange medical resources. The medical resource manager 270 may generate and output a recommendation based on the output of the model.

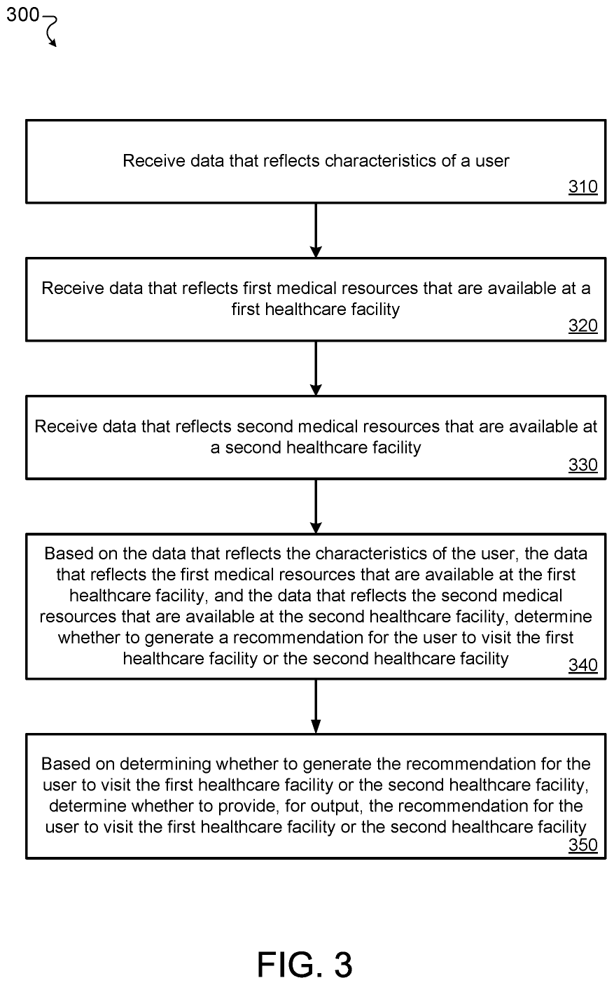

FIG. 3 is a flowchart of an example process 300 for generating a recommendation for a user to visit a healthcare facility based on data that reflects the characteristics of the user. In general, the process 300 receives data that reflects characteristics of a user and data that indicates the medical resources of various nearby healthcare facilities. Based on the user data and the medical resource data, the process 300 determines whether to recommend for the user to visit one of the healthcare facilities. The process 300 will be described as being performed by the server 106 of FIG. 1 and will include references to other components in FIG. 1. In some implementations, the process 300 may be performed by the server 200 of FIG. 2.

The server 106 receives data that reflects characteristics of a user (310). In some implementations, server 106 may receive the data that reflects the characteristic of a user from a quantified self device. A quantified self device may be a wearable device that is configured to detect health related data of a user. The health related data may include blood pressure data, pulse data, blood oxygen data, electrocardiogram data, breathing rate data, body temperature, and/or any other similar data. In some implementations, the data that reflects characteristics of the user may also include data collected from a computing device of the user. This data may reflect the environment of the user and include accelerometer data, location data, audio data, image data, proximity sensor data, ambient light data, gravity sensor data, gyroscope data, ambient temperature and/or any other similar data. In some implementations, the data that reflects characteristics of the user may include data provided from the user, such as weight data, height data, and/or other similar data.

The server 106 receives data that reflects first medical resources that are available at a first healthcare facility (320). The server 106 receives data that reflects second medical resources that are available at a second healthcare facility (330). In some implementations, the data that reflects medical resources that are located at and in use at a healthcare facility may include medications, vaccines, ventilators, blood pressure monitors, pulse oximeters, vital sign monitoring devices, and other similar devices. These may be examples of reusable medical resources. In some implementations, the data that reflects medical resources may include consumable medical resources that are available at the hospitals such as medications, vaccines, bandages, and any other similar type of medical resources that are not reused. In some implementations, the server 106 may receive data that indicates the usage rate of the consumable and reusable medical resources. In some implementations, the server 106 may receive data indicating a location of the healthcare facilities.

Based on the data that reflects the characteristics of the user, the data that reflects the first medical resources that are available at the first healthcare facility, and the data that reflects the second medical resources that are available at the second healthcare facility, the server 106 determines whether to generate a recommendation for the user to visit the first healthcare facility or the second healthcare facility (340). In some implementations, the healthcare recommendation generator 148 of the server 106 may analyze the data that reflects the characteristics of the user, the data that reflects the first medical resources that are available at the first healthcare facility, and the data that reflects the second medical resources that are available at the second healthcare facility using one or more rules and/or models. The rules and/or models may output data indicating whether to generate a recommendation for the user to visit the first healthcare facility or the second healthcare facility, for the user to quarantine, or for the user to take another type of action. In some implementations, the rules and/or models may output data indicating for the user to not visit the first healthcare facility or the second healthcare facility and to not quarantine.

In some implementations, the server 106 may access historical data that includes (i) previous data that reflects previous characteristics of various users at various healthcare facilities and nearby various healthcare facilities, and (ii) previous data that reflects medical resources that are available at the various healthcare facilities. The server 106 may train, using machine learning, a model. The server 106 may use the model to analyze the data that reflects the characteristics of the user, the data that reflects the first medical resources that are available at the first healthcare facility, and the data that reflects the second medical resources that are available at the second healthcare facility.

In the instances where the server 106 determines to generate a recommendation for the user to visit the first healthcare facility or the second healthcare facility, the server 106 may generate data indicating a health condition of the user based on the data that reflects the characteristics of the user. The server 106 may provide this data to the healthcare facility in order to provide the healthcare facility advanced notification of the possible visit by the user.

Based on determining whether to generate the recommendation for the user to visit the first healthcare facility or the second healthcare facility, the server 106 determines whether to provide, for output by the computing device, the recommendation for the user to visit the first healthcare facility or the second healthcare facility (350). If the rules and/or models output data indicating for the user to not visit the first healthcare facility or the second healthcare facility and to not quarantine, then the server 106 may bypass outputting the recommendation for the user to visit the first healthcare facility or the second healthcare facility or to quarantine.

In some implementations, the server 106 may receive data indicating whether the user intends to comply with the recommendation. In the case of the user indicating that the user will comply with visiting the healthcare facility, the server 106 may output the notification to the healthcare facility indicating that the user will visit and the condition of the user.

In some implementations, the server 106 may analyze the data that reflects the characteristics of the user, the data that reflects the first medical resources that are available at the first healthcare facility, and the data that reflects the second medical resources that are available at the second healthcare facility. Based on that analysis, the server 106 may determine that the first healthcare facility will likely need, in the future, one or more medical resources that the first healthcare facility may not have. The server 106 may determine that the second healthcare facility has the needed medical resources. In this case, the server 106 may generate and output a recommendation for the second healthcare facility to transfer those medical resources to the first healthcare facility.

In some implementations, the server 106 may analyze the data that reflects the characteristics of the user, the data that reflects the first medical resources that are available at the first healthcare facility, and the data that reflects the second medical resources that are available at the second healthcare facility and determine to recommend that the user visit the first or second healthcare facility. The server 106 may determine a likelihood that the user has a certain health condition, which may correspond to a confidence score that the user has the condition. The server may select the first or second healthcare facility based on the medical resources available at the facilities. If both have the needed medical resources to treat the condition, then the server 106 may select the facility closes to the user. If only one has the needed medical resources, then the server 106 may select that facility.

In some implementations, the server 106 may use the confidence score that the user has the condition to determine whether to recommend visiting a healthcare facility or quarantining. If the confidence score is in a particular range, then the server 106 may recommend quarantining. If the confidence score is above a threshold, then the server 106 may recommend visiting a healthcare facility. In some implementations, the ranges and thresholds may depend on the distance between the user and the healthcare facilities. If the distance between the user and the healthcare facilities is greater than a threshold distance, then the range to recommend quarantining may increase and the threshold to recommend visiting a healthcare facility may increase.

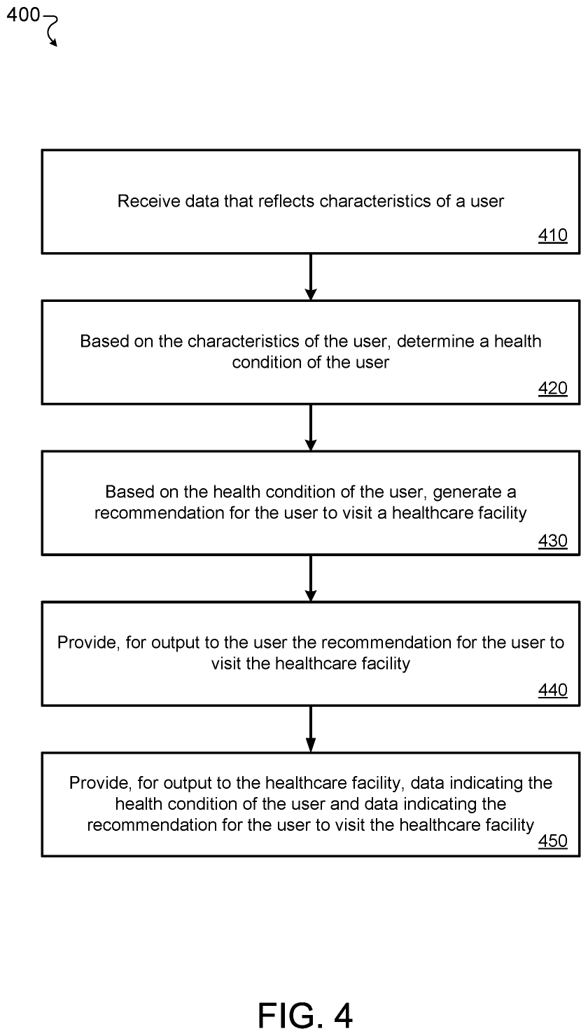

FIG. 4 is a flowchart of an example process 400 for providing a healthcare facility notification of a condition of a person who received a recommendation to visit the healthcare facility. In general, the process 400 receives data that reflects characteristics of a user. Based on the user data, the process 400 determines a health condition of the user and recommends that the user visit a healthcare facility. The process 400 generates and outputs a notification indicating the condition of the user and provides the notification to the healthcare facility. The process 400 will be described as being performed by the server 106 of FIG. 1 and will include references to other components in FIG. 1. In some implementations, the process 400 may be performed by the server 200 of FIG. 2.

The server 106 receives data that reflects characteristics of a user (410). In some implementations, server 106 may receive the data that reflects the characteristic of a user from a quantified self device. A quantified self device may be a wearable device that is configured to detect health related data of a user. The health related data may include blood pressure data, pulse data, blood oxygen data, electrocardiogram data, breathing rate data, body temperature, and/or any other similar data. In some implementations, the data that reflects characteristics of the user may also include data collected from a computing device of the user. This data may reflect the environment of the user and include accelerometer data, location data, audio data, image data, proximity sensor data, ambient light data, gravity sensor data, gyroscope data, ambient temperature and/or any other similar data. In some implementations, the data that reflects characteristics of the user may include data provided from the user, such as weight data, height data, and/or other similar data.

Based on the characteristics of the user, the server 106 determines a likely health condition of the user (420). The server 106 may analyze the data that reflects characteristics of the user using various models and/or rules. The models may be trained using machine learning and historical data that includes previous user data and the health conditions of those users. The rules may be automatically generated or user-specified and indicate patterns in the user data that may indicate certain health conditions. In some implementations, the server 106 may generate a confidence score that the user has the health condition.

Based on the health condition of the user, the server 106 generates a recommendation for the user to visit a healthcare facility (430). In some implementations, the server 106 may determine to recommend for the user to visit a healthcare facility based on the confidence score that the user has the health condition satisfying a threshold. In some implementations, the threshold may be based on the type of health condition. For example, if there is less than ninety-five percent chance that the user has bronchitis, then the server 106 may recommend quarantining and not generate a recommendation for the user to visit a healthcare facility. If there is greater than a twenty percent chance of a heatstroke, then the server 106 may generate a recommendation for the user to visit a healthcare facility.

The server 106 provides, for output to the user the recommendation for the user to visit the healthcare facility (440). The server 106 may output the recommendation to a computing device of the user. The user may provide an indication that the user will comply with the recommendation or reject the recommendation.

The server 106 provides, for output to the healthcare facility, data indicating the health condition of the user and data indicating the recommendation for the user to visit the healthcare facility (450). In some implementations, the server 106 outputs, to the healthcare facility, the data indicating the health condition of the user and data indicating the recommendation for the user to visit the healthcare facility in response to the user confirming that the user will comply with the recommendation.

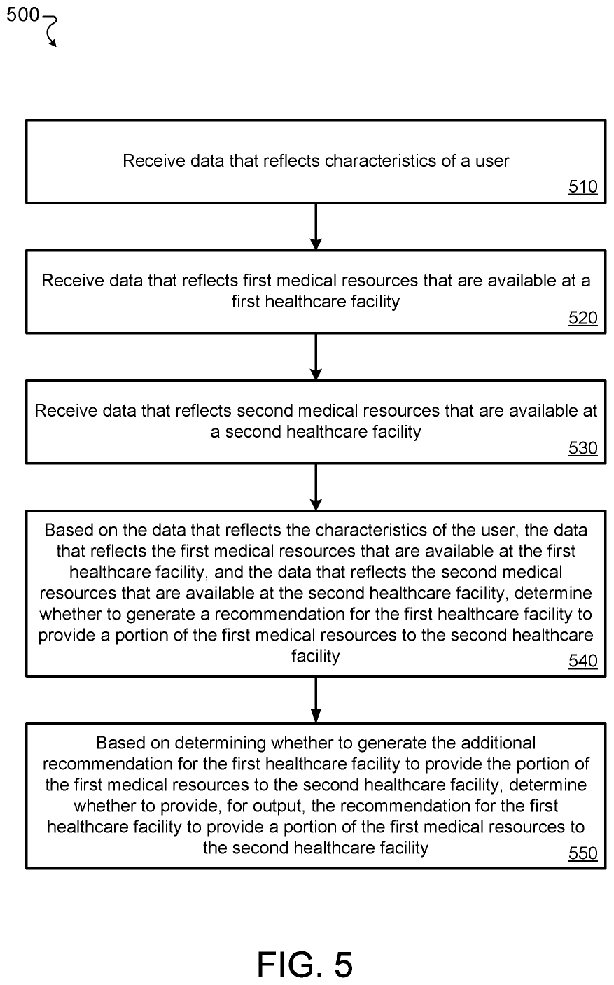

FIG. 5 is a flowchart of an example process 500 for generating a recommendation for a healthcare facility to transfer medical resources to another healthcare facility. In general, the process 500 receives data that reflects characteristics of a user and data that indicates the medical resources of various nearby healthcare facilities. Based on the user data and the medical resource data, the process 500 determines whether to recommend for one of the healthcare facilities to provide medical resources to another healthcare facility. The process 500 will be described as being performed by the server 106 of FIG. 1 and will include references to other components in FIG. 1. In some implementations, the process 500 may be performed by the server 200 of FIG. 2.

The server 106 receives data that reflects characteristics of a user (510). In some implementations, server 106 may receive the data that reflects the characteristic of a user from a quantified self device. A quantified self device may be a wearable device that is configured to detect health related data of a user. The health related data may include blood pressure data, pulse data, blood oxygen data, electrocardiogram data, breathing rate data, body temperature, and/or any other similar data. In some implementations, the data that reflects characteristics of the user may also include data collected from a computing device of the user. This data may reflect the environment of the user and include accelerometer data, location data, audio data, image data, proximity sensor data, ambient light data, gravity sensor data, gyroscope data, ambient temperature and/or any other similar data. In some implementations, the data that reflects characteristics of the user may include data provided from the user, such as weight data, height data, and/or other similar data. In some implementations, the server 106 may receive data that reflects characteristics of multiple users. These users may be in a same geographic area such as a same city or within a same radius that includes multiple healthcare facilities.

The server 106 receives data that reflects first medical resources that are available at a first healthcare facility (520). The server 106 receives data that reflects second medical resources that are available at a second healthcare facility (530). In some implementations, the data that reflects medical resources that are located at and in use at a healthcare facility may include medications, vaccines, ventilators, blood pressure monitors, pulse oximeters, vital sign monitoring devices, and other similar devices. These may be examples of reusable medical resources. In some implementations, the data that reflects medical resources may include consumable medical resources that are available at the hospitals such as medications, vaccines, bandages, and any other similar type of medical resources that are not reused. In some implementations, the server 106 may receive data that indicates the usage rate of the of the consumable and reusable medical resources. In some implementations, the server 106 may receive data indicating a location of the healthcare facilities.

Based on the data that reflects the characteristics of the user, the data that reflects the first medical resources that are available at the first healthcare facility, and the data that reflects the second medical resources that are available at the second healthcare facility, the server 106 determines whether to generate a recommendation for the first healthcare facility to provide a portion of the first medical resources to the second healthcare facility (540). The server 106 may determine that characteristics of the users may indicate a health condition of some of the users. That condition may require certain medical resources. If those users are near the second healthcare facility, then the server 106 may generate recommendations for those users to visit the second healthcare facility. If the second healthcare facility does not have the required medical resources, but the first healthcare facility does, then the server 106 may generate a recommendation to for the first healthcare facility to transfer those medical resources to the second healthcare facility.

Based on determining whether to generate the additional recommendation for the first healthcare facility to provide the portion of the first medical resources to the second healthcare facility, the server 106 determines whether to provide, for output, the recommendation for the first healthcare facility to provide a portion of the first medical resources to the second healthcare facility (550). In some implementations, the recommendation may include an option for the first healthcare facility to indicate whether the first healthcare facility will comply with the recommendation. If the first healthcare facility does not comply with the recommendation, then the server 106 may attempt to identify additional sources of the medical resources and/or recommend that the users visit the second healthcare facility.

Although a few implementations have been described in detail above, other modifications are possible. In addition, the logic flows depicted in the figures do not require the particular order shown, or sequential order, to achieve desirable results. In addition, other actions may be provided, or actions may be eliminated, from the described flows, and other components may be added to, or removed from, the described systems. Accordingly, other implementations are within the scope of the following claims.

What is claimed is:

1. A computer-implemented method, comprising:
receiving, by a computing device, data that reflects characteristics of a user;
receiving, by the computing device, data that reflects first medical resources that are available at a first healthcare facility;
receiving, by the computing device, data that reflects second medical resources that are available at a second healthcare facility;
based on the data that reflects the characteristics of the user, the data that reflects the first medical resources that are available at the first healthcare facility, and the data that reflects the second medical resources that are available at the second healthcare facility, determining, by the computing device, whether to generate a recommendation for the user to visit the first healthcare facility or the second healthcare facility; and
based on determining whether to generate the recommendation for the user to visit the first healthcare facility or the second healthcare facility, determining whether to provide, for output by the computing device, the recommendation for the user to visit the first healthcare facility or the second healthcare facility.

2. The method of claim 1, wherein determining whether to generate the recommendation for the user to visit the first healthcare facility or the second healthcare facility comprises:
providing, by the computing device, the data that reflects the characteristics of the user, the data that reflects the first medical resources that are available at the first healthcare facility, and the data that reflects the second medical resources that are available at the second healthcare facility as an input to a model; and
receiving, from the model and by the computing device, data indicating whether to generate the recommendation for the user to visit the first healthcare facility or the second healthcare facility.

3. The method of claim 2, comprising:
receiving, by the computing device, historical data that includes (i) previous data that reflects previous characteristics of various users at various healthcare facilities and nearby various healthcare facilities, and (ii) previous data that reflects medical resources that are available at the various healthcare facilities; and
training, by the computing device, the model using machine learning and the historical data.

4. The method of claim 1, wherein:
determining whether to generate the recommendation for the user to visit the first healthcare facility or the second healthcare facility comprises determining to generate the recommendation for the user to visit the first healthcare facility, and
determining whether to provide, for output, the recommendation for the user to visit the first healthcare facility or the second healthcare facility comprises determining to provide, for output, the recommendation for the user to visit the first healthcare facility.

5. The method of claim 4, comprising:
in response to determining to provide, for output, the recommendation for the user to visit the first healthcare facility, generating, by the computing device, data indicating a health condition of the user based on the data that reflects the characteristics of the user; and
providing, for output by the computing device, the data indicating the health condition of the user.

6. The method of claim 1, wherein:
determining whether to generate the recommendation for the user to visit the first healthcare facility or the second healthcare facility comprises determining to bypass generating the recommendation for the user to visit the first healthcare facility or the second healthcare facility, and
determining whether to provide, for output, the recommendation for the user to visit the first healthcare facility or the second healthcare facility comprises determining to bypass providing, for output, the recommendation for the user to visit the first healthcare facility or the second healthcare facility.

7. The method of claim 1, comprising:
based on the data that reflects the characteristics of the user, the data that reflects the first medical resources that are available at the first healthcare facility, and the data that reflects the second medical resources that are available at the second healthcare facility, determining, by the computing device, whether to generate an additional recommendation for the first healthcare facility to provide a portion of the first medical resources to the second healthcare facility; and
based on determining whether to generate the additional recommendation for the first healthcare facility to provide the portion of the first medical resources to the second healthcare facility, determining whether to provide, for output by the computing device, the additional recommendation for the first healthcare facility to provide a portion of the first medical resources to the second healthcare facility.

8. The method of claim 1, wherein receiving the data that reflects the characteristics of the user comprises receiving, from a quantified self device, the data that reflects the characteristics of the user.

9. The method of claim 1, wherein the first medical resources and the second medical resources comprise beds, medications, vaccines, ventilators, blood pressure monitors, pulse oximeters, and vital sign monitoring devices.

10. The method of claim 1, comprising:
selecting, by the computing device, the first healthcare facility and the second healthcare facility based on a location of the user, the data that reflects the characteristics of the user, the data that reflects the first medical resources that are available at the first healthcare facility, and the data that reflects the second medical resources that are available at the second healthcare facility.

11. The method of claim 1, comprising:
based on the data that reflects the characteristics of the user, the data that reflects the first medical resources that are available at the first healthcare facility, and the data that reflects the second medical resources that are available at the second healthcare facility, determining, by the computing device, whether to generate a recommendation for the user to quarantine; and
based on determining whether to generate the recommendation for the user to quarantine, determining whether to provide, for output by the computing device, the recommendation for the user to quarantine.

12. A system, comprising:
one or more processors; and
memory including a plurality of computer-executable components that are executable by the one or more processors to perform a plurality of actions, the plurality of actions comprising:
receiving, by a computing device, data that reflects characteristics of a user;
receiving, by the computing device, data that reflects first medical resources that are available at a first healthcare facility;
receiving, by the computing device, data that reflects second medical resources that are available at a second healthcare facility;
based on the data that reflects the characteristics of the user, the data that reflects the first medical resources that are available at the first healthcare facility, and the data that reflects the second medical resources that are available at the second healthcare facility, determining, by the computing device, whether to generate a recommendation for the user to visit the first healthcare facility or the second healthcare facility; and
based on determining whether to generate the recommendation for the user to visit the first healthcare facility or the second healthcare facility, determining whether to provide, for output by the computing device, the recommendation for the user to visit the first healthcare facility or the second healthcare facility.

13. The system of claim 12, wherein determining whether to generate the recommendation for the user to visit the first healthcare facility or the second healthcare facility comprises:
providing, by the computing device, the data that reflects the characteristics of the user, the data that reflects the first medical resources that are available at the first healthcare facility, and the data that reflects the second medical resources that are available at the second healthcare facility as an input to a model; and
receiving, from the model and by the computing device, data indicating whether to generate the recommendation for the user to visit the first healthcare facility or the second healthcare facility.

14. The system of claim 12, wherein:
determining whether to generate the recommendation for the user to visit the first healthcare facility or the second healthcare facility comprises determining to generate the recommendation for the user to visit the first healthcare facility, and
determining whether to provide, for output, the recommendation for the user to visit the first healthcare facility or the second healthcare facility comprises determining to provide, for output, the recommendation for the user to visit the first healthcare facility.

15. The system of claim 12, wherein:
determining whether to generate the recommendation for the user to visit the first healthcare facility or the second healthcare facility comprises determining to bypass generating the recommendation for the user to visit the first healthcare facility or the second healthcare facility, and
determining whether to provide, for output, the recommendation for the user to visit the first healthcare facility or the second healthcare facility comprises determining to bypass providing, for output, the recommendation for the user to visit the first healthcare facility or the second healthcare facility.

16. The system of claim 12, wherein the actions comprise:
based on the data that reflects the characteristics of the user, the data that reflects the first medical resources that are available at the first healthcare facility, and the data that reflects the second medical resources that are available at the second healthcare facility, determining, by the computing device, whether to generate an additional recommendation for the first healthcare facility to provide a portion of the first medical resources to the second healthcare facility; and
based on determining whether to generate the additional recommendation for the first healthcare facility to provide the portion of the first medical resources to the second healthcare facility, determining whether to provide, for output by the computing device, the additional recommendation for the first healthcare facility to provide a portion of the first medical resources to the second healthcare facility.

17. The system of claim 12, wherein:
receiving the data that reflects the characteristics of the user comprises receiving, from a quantified self device, the data that reflects the characteristics of the user, and
the first medical resources and the second medical resources comprise beds, medications, vaccines, ventilators, blood pressure monitors, pulse oximeters, and vital sign monitoring devices.

18. The system of claim 12, wherein the actions comprise:
selecting, by the computing device, the first healthcare facility and the second healthcare facility based on a location of the user, the data that reflects the characteristics of the user, the data that reflects the first medical resources that are available at the first healthcare facility, and the data that reflects the second medical resources that are available at the second healthcare facility.

19. The system of claim 12, wherein the actions comprise:
based on the data that reflects the characteristics of the user, the data that reflects the first medical resources that are available at the first healthcare facility, and the data that reflects the second medical resources that are available at the second healthcare facility, determining, by the computing device, whether to generate a recommendation for the user to quarantine; and
based on determining whether to generate the recommendation for the user to quarantine, determining whether to provide, for output by the computing device, the recommendation for the user to quarantine.

20. One or more non-transitory computer-readable media of a computing device storing computer-executable instructions that upon execution cause one or more computers to perform acts comprising:
receiving, by a computing device, data that reflects characteristics of a user;
receiving, by the computing device, data that reflects first medical resources that are available at a first healthcare facility;
receiving, by the computing device, data that reflects second medical resources that are available at a second healthcare facility;
based on the data that reflects the characteristics of the user, the data that reflects the first medical resources that are available at the first healthcare facility, and the data that reflects the second medical resources that are available at the second healthcare facility, determining, by the computing device, whether to generate a recommendation for the user to visit the first healthcare facility or the second healthcare facility; and
based on determining whether to generate the recommendation for the user to visit the first healthcare facility or the second healthcare facility, determining whether to provide, for output by the computing device, the recommendation for the user to visit the first healthcare facility or the second healthcare facility.
